(19) United States
(12) Patent Application Publication
(10) Pub. No.: US 2005/0205101 A1
(43) Pub. Date: Sep. 22, 2005

(54) Combined pharmocological and surgical method and system for the treatment of eye disorders

(76) Inventor: J. T. Lin, Oviedo, FL (US)

Correspondence Address: J. T. Lin, 4532 Old Carriage Trail, Oviedo, FL, 32765, US

(21) Appl. No.: 11/111,954

(22) Filed: Apr. 22, 2005

Related U.S. Application Data
(63) Continuation-in-part of application No. 10/271,992, filed on Oct. 17, 2002.

(51) Int. Cl. A61B 19/00; A61B 18/18

(52) U.S. Cl. 128/898; 606/4; 606/41

(57) ABSTRACT

The pharmacological means is to either “trigger” or enhance the contraction effect after a surgical method for larger accommodation and/or for more stable post-surgery results. The present invention also discloses the range of dose for both safety and effectiveness when it is used together with the surgical methods. The preferred embodiment for the surgical method to remove a portion of the soft tissue include lasers at wavelength of 0.19 to 0.36 um and 0.9 to 3.2 um and the non-laser device of radio frequency wave, electrode device, bipolar device and plasma assisted device. A deeper ablation depth of about 0.4 to 1.4 mm than the prior arts is disclosed. The preferred embodiment for pharmacological means includes the use of pilocarpine hydrochloride, phosphorothioate, physostigmine or other beta-adrenergic propanolamines.

### RELATED U.S. PAT. APPLICATION DATA

Continuation-in-part of Ser. No. 271,992, Oct. 17, 2002 which is now abandoned

### BACKGROUND OF THE INVENTION

1. Field of the Invention

The present invention relates to method and apparatus for the treatment of presbyopia and other eye disorders by changing the rigidity property of the sclera-ciliary complex by removing a portion of the sclera or ciliary tissue and treated by medication for minimum regression.

2. Prior Art

Presbyopia is a condition affects almost every individual with age over 45. Conventionally, this is treated by the use of additional convex lens spectacle for near work. The common method of correction for presbyopia is bifocal spectacle correction. In recent years attempts are being made to surgically reverse presbyopia The surgical methods include scleral expansion band (SEB), anterior ciliary sclerotomy (ACS), multi-focal intraocular lens (IOL) and the recent method using laser ablation of sclera tissue patented by the present inventor (U.S. Pat. Nos. 6,263,879 and 6,258,082) known as laser sclera ablation (LASA) or LAPR.

Restoring of accommodation achieved by SEB proposed by Schachar (U.S. Pat. Nos. 5,354,331, 5,489,299, 6,007,578) technique is very controversial with disagreement about the accommodative process and mechanisms at work. It was believed that patients after SEB may experience a pseudo-accommodation because of erosion caused by the implant with resultant scleral thinning, axial lengthening of the eye, myopic shift or the increase of spherical aberration and multifocality. Recent measurements of Mathews support the classical Helmholtz theory and refute Schachar's theory (Mathews S. “Scleral expansion surgery does not restore accommodation in human presbyopia”. Ophthalmology 1999; 106: 873-877). The techniques of SEB and ACS (Thornton, S, “Surgery for hyperopia and presbyopia”, edited by Neal Sher, Williams & Wilkins, MD, 1997, Chapter 4) are based on the concept of “lens crowding states” proposed by Schachar. This concept has not gained universal acceptance. Furthermore, clinical study has indicated major post-operative regression caused by tissue healing effects (Singh G, Chanlfin S. A complication of scleral expansion surgery for treatment of presbyopia. Am J Ophthalmol 2000; 130:521-523).

Prior art of Woodward (US Appl. No. 2002/0099363) proposed the use of thermal energy from a radiofrequency (RF) to treat presbyopia. However, there are no specifications of the RF disclosed, in which only low power RF can be used, if only thermal energy (rather than tissue ablation) is needed. In the present invention, we disclose the required parameter for a RF to be needed for tissue ablation.

The present inventor believes that the overall accommodation of an eye is governed by multiple factors and presbyopia may be caused by many of the ageing factors including the change of the ciliary and scleral tissues properties, the alteration in the elasticity, thickness and shape of the lens and its capsule and histological and physical changes in the scleral tissue and zonules. The laser presbyopia treatment (LPT) and the mechanism are based on a hypothesis presented as the “Lin-Kadambi hypothesis” (Lin and Kadambi, book chapter in Presbyopia: a Surgical Textbook, ed. by Agarwal et al, SLACK, N.J., 2002).

The “Lin-Kadambi” hypothesis proposed that after the LPT, the area of sclera ablated gets filled-in through the natural process of healing by “softer” sub-conjunctival tissue. The alteration in the elasticity of the tissue structure results in the ciliary body having to work against less resistance, a resistance initially caused by age-reduced rigidity of the sclera-ciliary-zonules complex. This leads to a greater relaxation of zonules and hence a greater central bulge of the crystalline lens for accommodation. This hypothesis may explain the minimal regression after LPT, however, can not explain some of the clinically reported cases with no accommodation effects after LPT.

The present inventor further proposed that the change in the elasticity of the sclera-ciliary-zonules complex provides a “dynamic” accommodation for patient to improve its near vision while the far vision remains, unlike the pseudo-accommodation effects provided by sclera expansion methods such as SEB, ACS and multifocal IOL.

For patients with “rigid” lens and/or ciliary body, the effectiveness of LPT may be very low due to the fact that the amount of ciliary-body contraction may not be sufficient to cause enough lens curvature change or anterior shift. Therefore, the present inventor proposes in this invention additional mechanism which uses pharmacological means to “trigger” or enhance the contraction effect after LPT for larger accommodation and/or for more stable post-operative results. Remove of sclera or ciliary tissue by a laser can be extended to the use of any means of tissue removal including other non-laser methods such as mechanical knife or electrode devices.

The prior art of Schachar, U.S. Pat. No. 5,503,165 (column 7, line 39-54) proposed a chemical treatment to weaken the sclera collagen to improve accommodation. Certain antibiotic drugs such as mitomycin are disclosed. However, there was no clinical data reported for Schachar's method which is fundamental different from what is proposed in the present invention using the cholinergic control for ciliary body contraction (to be detailed later). Since 1972, medicine methods have been reported to use the sympathetic innervations in human accommodation. The present invention proposes the combined effects of innervations and ablation of soft tissue of an eye. More details are discussed as follows.

Pharmacological methods for the studies of the role of sympathetic innervations in accommodation in humans has been reported in several prior arts. Rosenfield reported a study using an alpha-adrenergic antagonist caused an average increase in accommodative amplitude of 1.5 D, which however only maintain for less than 2 hours (Rosenfiled M, The influence of alpha-adrenergic agents on tonic accommodation”. Current Eye Research, vol. 9, No. 3, 1990, pp. 267-272).

Nyberg reported the use of Timolol, a beta-adrenergic antagonist to cause a net increase in tonic accommodation in unfocused eyes of a group of subjects with a mean age of 23. This effect has not been demonstrated in presbyopic patients (Nyberg G, “The Influence of beta-adrenoceptor agonists on accommodation of the Lens”, Clin. Exp. Pharmacol Physiol.; vol. 65, 1976; pp. 493-495). Beta-adrenergic antagonists such as timolol, betaxolol and levobunolol also have been used topically to control elevated intraocular pressure (IOP), where the beta-adrenergic antagonists were able to lower the IOP by decreasing the rate of production of aqueous humor by the ciliary body (van Alphen, “The adrenergic receptors of the intraocular pressure muscles of the human eye”, Invest. Ophthal. Vol. 15, 1976; pp. 502-505).

Eskridge reported a brief increase in the maximum accommodative response in a 36 year old subject treated with the parasympathomimetic drug eserine (Am. J. Optometry, August, 1972, pp. 632-635). A similar transient gain in accommodation was measured after treating subjects with the alpha-1 antagonist thymoxamine (Zetterstrom, Acta Ophthalmologica 65:699-704, 1987).

In a prior art of Neufeld (U.S. Pat. No. 5,488,050), vision of a 50 years old presbyopia was improved after administration of the eye by a beta-adrenergic antagonists of Timolol. However the long term results and accommodation amplitude were not disclosed.

Recently, Nolan (U.S. Pat. No. 6,273,092) reported the results of topical application of an acetylcholine esterase inhibitor to treat presbyopic patient. Acetylcholine esterase inhibitors such as (2-mercaptoethyl) trimethylammonium iodide O, O-diethyl phosphorothioate sold as PHOSPOHLINE IODIDE and physostigmine (also known as eserine) sold as ANTILIRIUM are commercially available and currently used for glaucoma and accommodative esotropia at a standard concentration of 0.03% to 0.25%. Nolan proposed to use a much lower concentration of 0.0075% to 0.12% to treat presbyopia. This prior art can improve near vision without side effect such as blurring, loss of distant vision or induction of myopia, which however only provides transient gain of accommodation and only lasts for (5-7) days.

The present inventor in U.S. Pat. Nos. 6,258,082 and 6,263,879 and PCT No. US01/24618 proposed the use of lasers to remove portion of sclera tissue and increase the elastic of sclera-ciliary-body complex to achieve near vision improvement for presbyopia patients, a procedure referred to as laser sclera ablation (LASA or LAPR). However, the clinical results of LASA showed post-operation regressions after 12-18 months in some cases. In addition, some reported cases showed no effects on subject's near vision due to un-known reasons. Based on the over 100 reported LASA cases, the mean accommodation improvement was only about 1.9 diopters which is not enough for those cases which have over 50% post-operation regression. Furthermore, for patients with “rigid” lens and/or ciliary body, the efficacy of LASA is poor due to the fact that the amount of ciliary-body contraction may not be sufficient to cause enough lens curvature change or anterior shift mainly due to the superficial effect of sclera ablation. Therefore, one objective of the present invention is to disclose a new mechanism which uses pharmacological or topical medicine method to “trigger” and enhance the “contraction” effect for higher accommodation and more stable results. In addition, the LASA (or LAPR) procedure is extended to a new procedure called laser ciliary-body ablation (LACA), which has a preferred total depth of about 0.4 to 1.4 mm, much deeper than that of LASA (limited to about 0.6 mm) and therefore LACA is more efficient than LASA (or LAPR). The preferred ablation also includes at least about 10% of ciliary body thickness. To avoid perforation, the preferred ablation area in LACA is about 0.5 to 1.5 mm away (outside) the limbus, which is narrower than that of LASA, about 0.5 to 4.5 mm.

It is known that there is an age correlation among glaucoma (open angle glaucoma) cataract formation and presbyopia. Also, there are glaucoma agents that actively stimulate the ciliary body to achieve pressure reduction in glaucoma. The LACA technique was also proposed by the present inventor for the treatment of glaucoma. Almost all post-LASA patients have a decrease of intraocular pressure (IOP) which however becomes to normal level within few days to few weeks after the LASA surgery. Therefore the LASA procedure is not a long-term effective method to reduce the IOP for glaucoma patients. It is yet another objective of this invention to disclose a long-term method to reduce the IOP of primary open angle glaucoma.

It is yet another objective of this invention to disclose the use of the similar mechanism based on an “elastic theory” in presbyopia correction for the new application of prevention, delay or reversal of AMD (age-related macular degeneration) by reducing their risk factors which includes choriodal low blood flow and the choroids capillary is high pressure. Increasing of ciliary body elasticity may also keep the lens flexible such that cataracts formation or protein disintegration may be prevented. Lens flexibility also prevents or delays amblyopia

So far, no attempt has been made to combine the use of a surgical method (such as removing sclera or ciliary-body tissue by a laser or other means) and the application of pharmacological means for stable, long-term and effective treatment of the above mentioned eye disorders including presbyopia, glaucoma, cataracts and AMD.

### BRIEF DESCRIPTION OF THE DRAWING

FIG. 1 shows the ablation area and preferred patterns.

FIG. 2 shows a comparison of prior art and this invention

### SUMMARY OF THE INVENTION

One preferred embodiment of this invention includes means and apparatus for increasing, enhancing and/or stabilizing the accommodation in presbyopia by the use of pharmacological means combined with a laser or non-laser surgery. Another preferred embodiment of this invention includes pharmacological means which utilizes the accommodation-enhancing effect under cholinergic control which includes the use of beta-adrenergic antagonist compounds or acetylycholine esterase inhibitors. It is yet another preferred embodiment to administrate the pharmacological means before or after a surgical method which removes portion of the sclera or ciliary-body tissue.

Another preferred embodiment of this invention includes lasers in the ultraviolet (UV) or infrared (IR) wavelength which matches the absorption spectra of the treated eye tissue.

Another preferred embodiment of this invention includes the ablation depth is about 0.4 to 1.4 mm, or most preferred 0.6 to 1.2 mm, in the area about 0.5 to 1.5 mm outside the limbus, having predetermined patterns of radial line, curve or dot ring.

The present invention also propose a mechanism which uses pharmacological means to “trigger” or enhance the contraction effect after a surgical method for larger initial accommodation and for more stable post-operative results. Another preferred embodiment of this invention includes surgical means of removal sclera or ciliary-body tissue by a laser or non-laser device such as electrode devices. Combining the surgical and pharmacological methods shall overcome the drawbacks of transient effect or post-treatment regression which occurs in procedure of prior arts, using only surgical or drugs. The methods disclosed herein can also be used to treat presbyopia and other disorders such as glaucoma, cataracts, amblyopia, age-related choroidal neovascularization (CNV) and AMD without adverse side effects by using controlled content percentage of the medicine.

Other features and advantages of this invention will be apparent from the following description and from the claims.

### DETAILED DESCRIPTION OF THE PREFERRED EMBODIMENTS AND DRAWINGS

The “Lin-Kadambi” hypothesis proposed that after laser presbyopia treatment (LPT), the area of sclera ablated gets filled-in through the natural process of healing by “softer” subconjunctival tissue. This essentially envisages that the effect of scleral ablative grooves created over the area overlying the ciliary body results in a favorable change in the elasticity of the sclera-ciliary-zonlues complex. This hypothesis may explain the minimal regression after LPT, however, can not explain some of the clinically reported cases with no accommodation effects after LPT.

To add a new postulate to the existing “Lin-Kadambi” hypothesis, the present inventor proposes the “Lin dynamic model” (see J. Refractive Surgery, vol. 20, 397-398, 2004) which goes a step further to propose that the total accommodation amplitude (AA), which is about 65% of the lens power change, has a non-linear response to the ciliary body contraction and may be achieved either by lens relaxation (LR) or by anterior shift (AS) or by combining LR and AS. In the present invention, we further propose that the change in the elasticity of the sclera-ciliary-zonules complex is a “dynamical” phenomena such that accommodation for patient to improve its near vision while the far vision remains, unlike the pseudo-accommodation effects provided by sclera expansion methods such as SEB, ACS and multifocal IOL.

For patients with “rigid” lens and/or ciliary body, the effectiveness of LPT may be small due to the fact that the amount of ciliary-body contraction may not be sufficient to cause enough lens curvature change or anterior shift. For “old” lens with less capsule elasticity, the ciliary body contraction may not cause the lens curvature change (which s required for accommodation). In this situation, anterior shift (AS) of the lens may dominate the mechanism of accommodation. Our calculation showed that each one mm AS may produce about (0.95-1.34) diopter of accommodation, depending on the lens curvatures.

In this invention, we propose additional preferred mechanism which uses pharmacological means to “trigger” or enhance the ciliary-body contraction effect after a surgical method for larger accommodation and/or for more stable post-operative results. In other words, the surgical method (SM) alone (as proposed by the prior arts of Lin) may not produce large enough accommodation for patient's need to read near, which typically requires a stable (1.5-3.0) diopters increase. On the other hand, the pharmacological means (PM) alone (as proposed in the prior arts of Neufeld and Nolan) produces only transient accommodation gain or a gain smaller than 1.5 diopters. We shall note that the medicine method of Schachar, U.S. Pat. No. 5,503,165, proposed the use of antibiotic drugs to weaken the scleral tissue is different in mechanism, comparing to the PM used in this invention which uses the cholinergic control for ciliary-body contraction (to be detailed later). To achieve sufficient and stable accommodation, say larger than 2.0 diopters, we propose in this invention a SM followed by a PM. In addition, the PM may also provide a long term stable accommodation without suffering regressions which occur after a SM without post-operative PM. The PM may also be applied before the SM to trigger or enhance the accommodation. Based on Lin-Kadambi theory, the refilling of sub-conjunctival to the ablated gap is essential for the elasticity increase of ciliary and scleral tissue. Therefore, the triggering (pre-operative) and enhancing (post-operative) by PM may also prevent the closing of the “gap” during the healing process.

Our reported clinical cases based on prior arts of Lin, U.S. Pat. Nos. 6,258,082 and 6,263,879 (referred to as Lin-082-879) showed that about 10% to 20% presbyopia subjects did not achieve the desired accommodation. For example, the post-operation Jeagar (J) reading remains as J5-J7, the same as that of pre-operation, where small J readings of J1-J3 means high accommodation. These poor results cases may be due to the “rigidity” of the lens or ciliary body which require a pharmacological means applied either before, during or after the surgical method in order to “trigger” the contraction effects and achieve desired accommodation to read J1-J3 for their near vision. For patients with more elastic lens capsule or mobility of the ciliary body, the surgical method typically will achieve a J reading of J2-J3 and accommodation average of about 1.9 diopter. In this case, the pharmacological means (PM) proposed in the present invention may enhance and/or stabilize the post-surgical results. Based on our more than 100 reported LASA (or LAPR) cases, the mean accommodation improvement was about 1.9 diopters which may not be enough for those cases with a 50% post-operation regression. Therefore enhancement and stabilization of the surgical method are highly desired and it is achievable by PM proposed in the present invention. Remove of sclera tissue by a laser referred as LASA can be extended to the use of any means of tissue removal including other non-laser methods such as mechanical knife or electrode devices, radiofrequency wave, etc.

Furthermore, low efficacy and regression of Lin-082-879 are due to the limitation of a maximal ablation depth of about 0.5 mm, or 80% to 90% of the sclera thickness. The present invention proposes a much deeper ablation into the ciliary-body (CB) area The preferred ablation depth is about 0.4 to 1.4 mm and most preferable about 0.6 to 1.2 mm including the ablation of conjunctiva, sclera, choroids or a portion of CB thickness. In this invention, we refer the new method of CB ablation by a laser or non-laser device as LACA, versus LASA in Lin's prior arts.

The ciliary muscle controls the shape of the lens and thereby causes the accommodation for a presbyopic patient to see near. The ciliary muscle has a dual innervations, receiving both sympathetic and parasympathetic fibers. Contraction of ciliary body necessary for accommodation is under parasympathetic (cholinergic) control and opposing cholinergic control, the sympathetic (adrenergic) innervations, which plays a minor role, is responsible for relaxation of the ciliary muscle or inhibition of accommodation (Gilmartin B., “A review of the role of sympathetic innervations of the ciliary muscle in ocular accommodation”, Optometry and Vision Science, vol. 69, 1992; pp. 276-282).

It is known that there is an age correlation among glaucoma (open angle glaucoma), cataract formation and presbyopia. Also, there are glaucoma agents that actively stimulate the ciliary body to achieve pressure reduction in glaucoma. Furthermore, there are theories that in glaucoma, lens proteins disintegrate and/or the ciliary body secretes proteins abnormally. Keeping the lens flexible through accommodation, therefore may prevent the disintegration of lens proteins (cataract formation). The LASA technique was also proposed by the present inventor for the treatment of glaucoma Almost all post-LASA patients have a decrease of intraocular pressure (IOP) which however becomes to normal level within few days to few weeks after the surgery. Therefore the LPT procedure is not an long-term effective method to reduce the IOP for glaucoma patients, if no follow-up PM is used.

The pathogenesis of AMD is not entirely known. The vascular model proposed by E. Friedman (Am J Ophthalmol vol. 130, pp. 658-663 2000) stated that AMD is the result of the accumulation of lipid in the sclera and in Bruch Membrance, progressively increasing the stiffness of these tissues and increasing the postcapillary resistance of the choroidal vasculature, situated between the progressively noncompliant sclera and noncompressable contents of the globe. This model also stated that in addition to decreasing choroidal blood flow, the increase in resistance or elevation the hydrostatic pressure of the choriocapillaris, enhancing leakage and deposition of extracellular proteins and lipids. In AMD, the location of the lipid deposition is also a function of the intravascular hydrostatic pressure. The lipids deposited in the sclera may originate in scleral vessels or they may reach the sclera from the choroids by diffusion or filtration down the transsclera hydrostatic pressure gradient. In addition, it was also reported that hyperopia is frequently identified as a risk factor for AMD in large case-control epidemiological studies (Tang et al, German J Opthalmol 1993; vol. 2, pp. 10-13). The vascular model of Freidman suggested that this can be attributed to the increased scleral rigidity associated with hyperopia.

The compounds useful in practicing pharmacological means in this invention shall include any beta-adrenergic antagonists which produce a net sympatholytic response, resulting in increased accommodation, by binding to beta-adrenergic receptors within the ciliary muscle of the eye. Without limiting the invention to the specific groups and compounds listed, the following is a list of representative beta-adrenergic antagonists useful in this invention and was patented in prior arts: Acebutolol (U.S. Pat. No. 3,857,952), (U.S. Pat. No. 4,217,305), Arotinolol (U.S. Pat. No. 3,932,400), Atenolol (U.S. Pat. Nos. 3,663,607 and 3,836,671), Befunolol (U.S. Pat. No. 3,853,923), Betaxolol (U.S. Pat. No. 4,252,984), Bevantolol (U.S. Pat. No. 3,857,891), Bisoprolol (U.S. Pat. Nos. 4,171,370 and 4,258,062), Bopindolol (U.S. Pat. No. 4,340,541), Bucumolol (U.S. Pat. No. 3,663,570), Bufetolol (U.S. Pat. No. 3,723,476), Bufuralol (U.S. Pat. No. 3,929,836), Bunitrolol (U.S. Pat. Nos. 3,940,489 and 3,961,071), Bunolol HCl (also known as levobunolol) 1(2H)-Naphthalenone,5-[3-1,(1-dimethylethyl)amino}-2-hydroxypropoxy]-3,4-dihydro,hydrochlorie (+)(U.S. Pat. No. 3,649,691 and U.S. Pat. No. 4,463,176), Bupranolol (U.S. Pat. No. 3,309,406), Butofilolol (U.S. Pat. No. 4,252,825), Carteolol (U.S. Pat. No. 3,910,924), Carvedilol (U.S. Pat. No. 4,503,067), Cetamolol (U.S. Pat. No. 4,059,622), Epanolol (U.S. Pat. No. 4,167,581), Esmolol (U.S. Pat. No. 4,387,103), Indenolol (U.S. Pat. No. 4,045,482), Labetalol (U.S. Pat. No. 4,012,444), Mepindolol (Swiss Patents 469,002 and 472,404), Metoprolol (U.S. Pat. No. 3,873,600), Moprolol (U.S. Pat. No. 3,501,769), Nadolol (U.S. Pat. No. 3,935,267), Nadoxolol (U.S. Pat. No. 3,819,702), Nifenalol (British Patent 950,682), Nipradilol (U.S. Pat. Nos. 4,394,382 and 4,727,085), Penbutolol (U.S. Pat. No. 3,551,493), Practolol (U.S. Pat. No. 3,408,387), Propranolol (U.S. Pat. Nos. 3,337,628 and 3,520,919, Talinolol (U.S. Pat. Nos. 3,935,259 and 4,038,313), Tertatolol (U.S. Pat. No. 3,960,891), Timolol (U.S. Pat. Nos. 3,655,663 and 3,657,237), Toliprolol (U.S. Pat. Nos. 3,432,545 and 3,459,782), and Xibenolol (U.S. Pat. No. 4,018,824).

Some of the above beta-adrenergic propanolamines are also known in the Merck Index, Unlisted Drugs, USAN and USP Dictionary of Drug Names, and Annual Reports in Medicinal Chemistry, Vol. 10, pages 51-60 (1975), and ibid., Vol. 14, pages 81-90 (1979).

Another preferred compound is the cholinesterase inhibitor, such as phospholine iodide, but administered in a more diluted concentrations preferred to be about 0.01% to 0.3%. Phospholine iodide is currently used for glaucoma and accommodative esotropia but there has been no successful use of this drug for presbyopia because of many adverse side effects of the drug when used in the standard doses established for glaucoma and accommodative esotropia. Prior art of Nolan (U.S. Pat. 6,273,092) proposed low concentration phospholine iodide, 0.001% to 0.25%, for the treatment of presbyopia. This prior art can improve near vision without side effect such as blurring, loss of distant vision or induction of myopia, which however only provides transient gain of accommodation and only lasts for about (5-7) days. In the present invention, we prose the use of the low concentration phospholine either before or after the surgical method which removes a portion of the sclera or ciliary-body tissue such that the combined means achieves stable and efficient accommodation.

Another yet preferred compound is the pilocarpine hydrochloride, an acetylcholine like drug, sold as SALAGER.RTM. (MGI Pharma, Minnetonka, Minn.). Pilocarpine hydrochloride at typical concentration of about 4% has been used to an emmetropic eye, the increased parasymathetic effect leads to enhanced near vision but at the sacrifice of distant vision. The emmetropic eye becomes myopic as a consequence of this adverse side effect, thus acetylcholine treatment to correct presbyopia has not been effective. However, we propose in this invention a lower concentration of about (0.5% -5%) and most preferable about 0.5% to 2% of pilocarpine used for patients only combined with the presbyopia surgical method, either before or after the surgery.

Formulations of the invention include any formulation in which the compounds of the invention may be delivered to the eye. One of the preferred embodiments is in a topical preparation which is adapted to be applied to the surface of the eye. Such preparations usually have liquid carriers which can be aqueous solutions or suspensions. The compounds of the invention are administered in therapeutically effective amounts. A therapeutically effective amount is one which causes medically useful increase in accommodative ability of a presbyopic eye. Such an increase is at least 1.0 and preferably 1.5 diopter.

In one preferred embodiment of the invention, the compounds are administered before the surgery, or right after the surgery or the bedtime after the surgery. Depending on the progress of the post-surgery patients, administration of the proposed compounds may be (1-2) times per day for a period of (1-60) days after the surgical method, or administered only when post-operation regression starts. Administration of the compounds before the surgery is preferred to “trigger” the accommodation and most preferable for senior patients with age over (55-60), or for patients with presbyopia diopter over +4.0. As discussed earlier, the purpose of using pharmacological means is to either “trigger” or enhance the contraction effect after a surgical method for larger accommodation and/or for more stable post-surgery results. In addition, the invention discloses that the preferred lower dose range is especially useful in providing eye drugs that is low enough to be both safe and effective when used together with the surgical methods.

The preferred embodiment for the surgical method to remove a portion of the soft tissue of an eye includes lasers with wavelength of (190 to 360) nm, (970 to 1600) nm or (2.6 to 3.2) microns and the non-laser methods such as physical blades or knife, electromagnetic wave such as radio frequency (RF) wave, electrode device, bipolar or monopolar tip device and plasma assisted electrode device. The electromagnetic wave generator is commercially available. However, the parameters of the device such as its frequency, pulse duration and repetition rate and the size of the electrode tip shall be selected for efficient cutting (or ablation) with minimum thermal damage to the tissue to be removed. The preferred embodiments of the lasers include: harmonics of Nd:YAG, Nd:YVO4, or Nd:YLF (at UV of 212 to 266 nm) laser, Er:YAG (at 2.94 um), Er:YAGG, Er:Cr:YSGG, Er:Cr:Th:YAG or Er:YLAO3 (at about 2.7 to 2.8 um) excimer lasers (at 193, 248, 308 nm), diode lasers at (0.95-1.9) um, and Ho:YAG (at about 2.1 um).

The preferred RF or electrode device includes a frequency of about 20 KHz to 1,000 KHz, power of about 0.5 to 20 W, and end-tip (contacting the treated surface) size of about 0.2 to 5 mm.

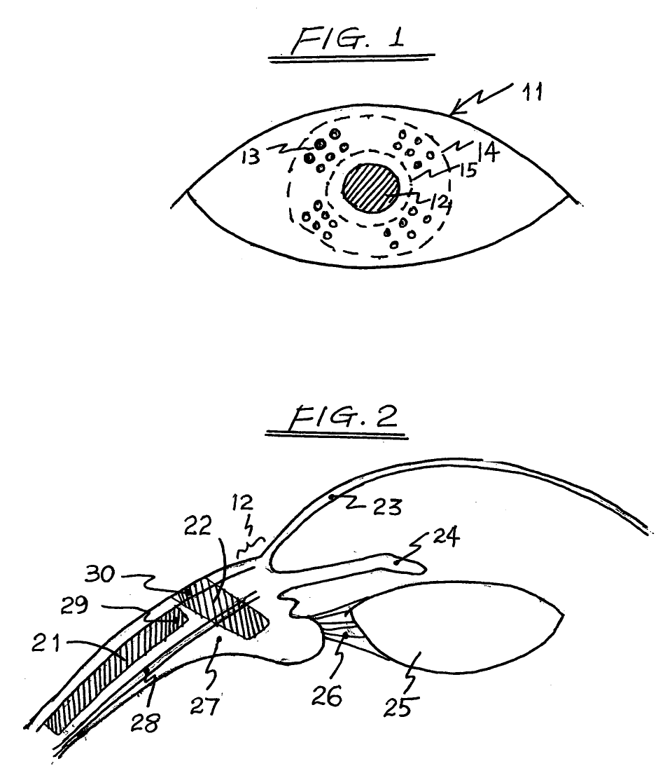

As shown in FIG. 1, the ablation is outside the limbus 12 of an eye 11 and within the area defined by two circles 14 and 15 having a diameter of about 13 and 11 mm, respectively. We note that the circle 14 has a larger diameter 18 mm in the prior arts of Lin which limits the ablation depth. The preferred ablation patterns 13 includes radial line, curve, ring dots or any non-specified patterns and having an ablation depth of about 0.4 to 1.4 mm and most preferable of about 0.6 to 1.2 mm, which is deeper than the prior arts of Lin-082-879, 0.4 to 0.6 mm. For minimal invasive, the most preferable pattern is dotted rings surrounding the area about 0.5 to 1.5 mm outside the limbus and each dot has a preferred diameter of about 0.5 to 1.5 mm. At least 8 dots in each treated-eye is preferred and most preferable about 12 to 24 dots.

The preferred ablation area of this invention is much narrower than that of Lin's prior art which is 0.5 to 4.5 mm outside the limbus and suffers the risk of perforation. The present invention is much safer due to the protection of the beneath ciliary-body (about 1.0 to 1.5 mm in thickness), and more effective due to the deeper ablation. A comparison of the ablation area and depth are shown in FIG. 2, where ablation area of the present invention 22 has a much deeper ablation depth than that of prior arts of Lin-082-879, shown by area 21. Also shown in FIG. 2 is the cornea 23, iris 24, lens 25 which is connected to the ciliary-body 27 by the zonules fiber 26. Above the ciliary-body is the choroids 28, sclera 29 and conjunctival layer 30.

The total accommodation short after the procedure using the medicine shall include the tissue removal effects and the effect due to medicine. Long terms stable results are attributed mainly to deep tissue removal which is further enhanced by the medicine. The initial ciliary contraction by medicine is important for stable long term results which require minimizing the regression caused by tissue healing, particularly for the period of about 2 to 5 days for sub-conjunctiva filling to complete.

While the invention has been shown and described with reference to the preferred embodiments thereof, it will be understood by those skilled in the art that the foregoing and other changes and variations in form and detail may be made therein without departing from the spirit, scope and teaching of the invention. Accordingly, threshold and apparatus, the ophthalmic applications herein disclosed are to be considered merely as illustrative and the invention is to be limited only as set forth in the claims.

1. A method for treating an eye disorder which comprises the steps of:
(a) selecting a soft tissue removal means;
(b) controlling said soft tissue removal means to remove a portion of soft tissue of an eye in a predetermined area, pattern and depth of the eye; and
(c) selecting a pharmacological means administrating to the treated-eye,
wherein said eye disorder includes presbyopia, amblyopia, glaucoma, cataracts, choroidal neovascularization, or age-related macular degeneration (AMD).

2. The method of claim 1, wherein said predetermined area is an area about 0.5 to 1.5 mm outside the limbus of an eye, having a preferred said predetermined depth about 0.4 to 1.4 mm and most preferable about 0.6 to 1.2 mm, and having a preferred said predetermined pattern of radial line, curve, dotted rings or non-specified patterns.

3. The method of claim 1, wherein the amblyopia is treated or prevented by keeping the lens flexible through the increase of accommodative ability of the eye.

4. The method of claim 1, wherein the glaucoma is treated or prevented by reduction of the intraocular pressure caused by increasing of contraction ability of said ciliary body.

5. The method of claim 1, wherein the cataracts formation or disintegration of lens proteins is prevented by keeping the lens flexible through accommodation.

6. The method of claim 1, wherein the choroidal neovascularization caused by degenerative myopia or age-related macular degeneration (AMD) is prevented or treated by the increasing of contraction ability or decreasing of rigidity of sclera and ciliary muscle.

7. The method of claim 1, wherein the near vision of presbyopia is improved by the increasing of the accommodation achieved by increasing of contraction ability of said ciliary-body.

8. The method of claim 7, wherein increasing of said contraction ability is partially caused by the increasing of the elasticity of said cilciary-body after a portion of said soft tissue is removed, wherein said soft tissue includes sclera, conjunctiva, choroids or ciliary-body of an eye.

9. The method of claim 7, wherein said near vision of said presbyopia is further improved or stabilized by the increase of said contraction ability of said ciliary body achieved by said pharmacological means.

10. The method of claim 7, wherein said near vision of said presbyopia increases by at least 1.0 diopters and the improved said near vision remains at least 6 months post treatment.

11. The method of claim 1, wherein said tissue removal means includes tissue removed by a device selected from the group consisting of: laser, plasma knife, electromagnetic wave, radial frequency wave, and electrode device.

12. The method of claim 11, wherein said laser includes harmonic of Nd:YAG, Nd:YVO4, or Nd:YLF having output wavelength of about 212 to 266 nm, Er:YAG (at 2.94 micron), Er:Cr:YSGG, Er:YSGG or Er:YLO3 (at about 2.8 micron), excimer lasers (at 193, 248, 308 nm), diode laser (at about 0.95 to 2.1 micron), or Ho:YAG laser (at about 2.1 micron).

13. The method of claim 11, wherein said electromagnetic wave device has a radio frequency ranging of about 20 KHz to 1000 KHz and power of about 0.1 to 20 W.

14. The method of claim 11, wherein said electrode device includes a monopolar-tip, bipolar-tip, or plasma assisted electrode device operated at said ratio frequency.

15. The method of claim 1, wherein said pharmacological means includes topically administering to the eye an amount of a composition sufficient to further increase said accommodative ability of the subject by at least 0.5 diopters for near vision.

16. The method of claim 15, wherein the increase of said accommodative ability is caused by a parasympathetic (cholinergic) control of the ciliary muscle, whereby contraction of the ciliary body allows the zonules to relax and change the lens curvature for near vision.

17. The method of claim 1, wherein said pharmacological means includes topically administering to the eye an amount of a composition sufficient to minimize the post-operative regression of said accommodative ability of the subject without affecting distant vision.

18. The method of claim 17, wherein the post-operative regression of said accommodative ability of the subject is minimized by a parasympathetic (cholinergic) control of the ciliary muscle.

19. The method of claim 1, wherein said pharmacological means includes topically administering to the eye a composition having phosphorothioate or physostigmine content of about 0.01% to 0.3%.

20. The method of claim 1, wherein said pharmacological means includes topically administering to the eye a composition having pilocarpine hydrochloride with a preferred content about 0.2% to 5% and most preferable about 0.5% to 2%.

21. The method of claim 1, wherein said pharmacological means includes topically administering to the eye a composition having the beta-adrenergic antagonist selected from the group consisting of:
Acebutolol, Alprenolol, Amosulalol, Arotinolol, Atenolol, Befunolol, Betaxolol, Bevantolol, Bisoprolol, Bopindolol, Bucumolol, Bufetolol, Bufuralol, Bunitrolol, Bunolol HCl, Bupranolol, Butidrine HCl, Butofilolol, Carazolol, Carteolol, Carvedilol, Celiprolol, Cetamolol, Cicloprolol HCl Cloranolol, Dexpropranolol, Diacetolol HCl, Dilevalol, Epanolol, Esmolol, Exaprolol, Flestolol Sulfate, Indenolol, Labetalol, Mepindolol, Metalol HCl, Metoprolol, Moprolol, Nadolol, Nadoxolol, Nifenalol, Nipradilol, Oxprenolol, Pamatolol Sulfate, Penbutolol, Pindolol, Practolol, Pronethalol, Propranolol, Sotalol, Sulfinalol, Talinolol, Tertatolol, Timolol, Tiprenolol HCl, Tolamolol, Toliprolol, and Xibenolol.

22. The method of claim 1, wherein said pharmacological means includes topically administering to the eye a composition, wherein the composition is selected and is administered in an amount whereby the treatment of presbyopia is free of medically unacceptable side effects including elevated intraocular pressure, change of distant vision or myopic shift of the eye.

23. The method of claim 1, wherein said pharmacological means is administered before said tissue removal means.

24. The method of claim 1, wherein said pharmacological means is administered after said tissue removal means.

25. An apparatus for treating an eye disorder which comprises of:
(a) a tissue removal device to remove a portion of soft tissue outside the limbus of an eye in a predetermined area, pattern and depth; whereby the contraction ability of the ciliary-body; and
(b) a pharmacological product administrating to the subject;
wherein the disorder includes presbyopia, amblyopia, glaucoma, cataracts, choroidal neovascularization or age-related macular degeneration (AMD).

26. The apparatus of claim 25, wherein said tissue removal device is a device selected from the group consisting of laser, electromagnetic wave at radio frequency, electrode device, monopolar device, bipolar device, and plasma assisted electrode device.

27. The apparatus of claim 25, wherein said laser includes harmonic of Nd:YAG, Nd:YVO4, or Nd:YLF having output wavelength of about 212 to 266 nm, Er;YAG (at 2.94 micron), Er:YSGG, Er:Cr:YSGG, Er:Cr:Th:YAG or Er:YALO3 at about 2.7 to 2.8 micron), excimer lasers (at 193, 248, 308 nm), diode laser (at about 0.95 to 2.1 micron), or Ho:YAG laser (at about 2.1 micron).

28. The apparatus of claim 25, wherein said predetermined area is an area about 0.5 to 1.5 mm outside the limbus of an eye, having a preferred depth about 0.4 to 1.4 mm and most preferable about 0.6 to 1.2 mm, and having a preferred pattern of radial line, curve, dotted rings or non-specified patterns.

29. The apparatus of claim 25, wherein said pharmacological product includes a composition having phosphorothioate or physostigmine content of about 0.01% to 0.3%, or pilocarpine hydrochloride content of about 0.2% to 5% and most preferable of about 0.5% to 2%.

30. The apparatus of claim 25, wherein said pharmacological product includes a tropical composition of beta-adrenergic antagonist content of about 0.01% to 10% by weight.

31. The apparatus of claim 25, wherein said pharmacological product is administered before or after said soft tissue of an eye is removed, whereby patient's post-operative outcome is further enhanced or stabilized.

32. The apparatus of claim 25, wherein said soft tissue includes conjunctiva, sclera, choroids or ciliary-body of an eye.
